(19) United States
(12) Patent Application Publication
(10) Pub. No.: US 2020/0339974 A1
(43) Pub. Date: Oct. 29, 2020

(54) CELL LABELLING, TRACKING AND RETRIEVAL

(71) Applicant: Cancer Research Technology Ltd., London (GB)

(72) Inventors: Gregory Hannon, London (GB); Clare Rebbeck, London (GB); Simon Knott, Los Angeles, CA (US)

(73) Assignee: Cancer Research Technology Ltd., London (GB)

(21) Appl. No.: 16/487,745

(22) PCT Filed: Feb. 22, 2018
(86) PCT No.: PCT/EP2018/054450

§ 371 Date: Aug. 21, 2019

(30) Foreign Application Priority Data
Feb. 22, 2017 (GB) 1702847.3

(51) Int. Cl. C12N 15/10; C12N 15/86; C12N 15/11

(52) U.S. Cl. CPC C12N 15/102; C12N 15/86; C12N 2310/20; C12N 15/1086; C12N 2740/10043; C12N 15/11

(57) ABSTRACT

The present invention provides a method for targeted cell retrieval, comprising: providing a population of barcoded cells, said population comprising a plurality of different barcodes, each of the plurality of different barcodes being uniquely targetable with a target-specific CRISPR RNA; introducing a CRISPR-Cas system, or one or more vectors encoding the components of the CRISPR-Cas system, into the population of barcoded cells said CRISPR-Cas system having a target-specific CRISPR RNA that targets a first barcode of said plurality of different barcodes, thereby causing a CRISPR-Cas system-mediated change at a target site leading to a change in one or more detectable properties of at least one cell carrying said first barcode; and retrieving said at least one cell carrying said first barcode based on the change in said one or more detectable properties. Also provided are products and kits for use in the method of the invention.

This application claims priority from GB1702847.3 filed 22 Feb. 2017, the contents and elements of which are herein incorporated by reference for all purposes.

### FIELD OF THE INVENTION

The present invention relates to methods of cell tracking and target-specific cell retrieval.

### BACKGROUND TO THE INVENTION

The present invention is directed at methods, and related products and kits, for targeted cell retrieval, e.g., of barcoded cells from heterogeneous cell populations.

Cells from a common ancestor with identical genetic features are known as cellular clones. Despite historically the perception being that tumours and cell lines were clonal, it is now clear that highly heterogenous cell populations exist within a tumour, or within cell lines, and these have different mutation profiles, epigenetic changes, and gene expression profiles, leading to differing phenotypic properties. Monitoring clonal dynamics within heterogeneous cellular populations is important for multiple areas of biomedical research, including and not limited to, stem cell and cancer biology. Tracking the contributions of individual cells within large populations however, has been constrained by limitations in sensitivity and complexity. A recent approach that circumvents this shortcoming, combines viral cellular labelling, DNA barcoding and next-generation sequencing to monitor entire cell populations using a barcode system that scales to many thousands or even a million individual cells. The cell-tracking process begins with the introduction of a packaged viral library encoding a highly heterogeneous population of barcodes into a population of founder cells. After selection, treatment, or differentiation, barcode representation (assessed by next-generation sequencing) in cells provides data on which clones from the initial population survived, thrived, or died out.

While there are previously described examples of research groups using barcoding to track heterogeneous populations (Bhang H. E., et al., Nat. Med., 2015, Vol. 21, No. 5, pp. 440-448) and commercially available retroviral or lentiviral libraries with barcodes for cell tracking (Cellecta, Inc., Mountain View, Calif.), barcoding has not generally been used in reverse from in vivo heterogeneous cellular populations back to the original contributing in vitro clonal cell populations to provide a source of the identified cells for further experimental analyses, nor has it been possible to compare the desired clonal cell(s) with its less successful counterparts (due to these not being present in the population after experimental selection).

Wagenblast E. et al., Nature, 2015, Vol. 520, No. 7547, pp. 358-362, describe a cell-tracking process to follow genetically modified cancer cells in a polyclonal context throughout each stage of metastatic disease progression. In this paper the authors created a heterogeneous population from a panel of single cell-derived clones, which retained samples of individual clones. This illustrates that to be able to go back to clones that arise under experimental selection the user would need samples of the pure individual clones to go back to and that while this is feasible in this limited example, it would be both laborious and expensive if the user wanted to retain the ability to retrieve source clones for later biological analysis from a library of tens of thousands or millions of clones, because the user would need to sort, plate, expand and maintain large numbers of clonal cell populations before infection with corresponding individual barcodes. Indeed, if going down this laborious route, the cells maintained in clone bank would have to have the barcodes in them so that the user would be able to know which clone in the stored bank correlated with the clone identified from the experiment. Alternatively, the user would need to have a coded database linking well/vial number to a specific barcode sequence. In either case, this approach is clearly sub-optimal when employed at larger scale.

Key to the application of any genetic manipulation technology is efficiency.

The CRISPR-Cas9 genome-editing method is derived from a prokaryotic RNA-guided defence system. There are at least eleven different CRISPR-Cas systems, which have been grouped into three major types (I-III). In the type I and II systems, nucleotides adjacent to the protospacer in the targeted genome comprise the protospacer adjacent motif (PAM). The PAM is essential for Cas to cleave its target DNA. Type II CRISPR-Cas systems have been adapted as a genome-engineering tool. In this system, crRNA teams up with a second RNA, called trans-acting CRISPR RNA (tracrRNA), which is critical for crRNA maturation and recruiting the Cas9 nuclease to DNA. The RNA that guides Cas9 uses a short (˜20-nt) sequence to identify its genomic target. This three-component system was simplified by fusing together crRNA and tracrRNA, creating a single chimeric “guide” RNA (abbreviated as sgRNA or simply gRNA). Hybridisation of the sgRNA with the target sequence leads to cleavage of the target DNA at an adjacent/upstream PAM site and the cellular repair of the DNA break can lead to the insertion/deletion/mutation of bases and mutation at the target locus. The use of the common Cas9 nuclease in conjunction with multiple gRNAs to introduce mutations in several genes simultaneously has been carried out in cultured mammalian cells as well as genetic model organisms such as mice, zebrafish, and Arabidopsis (Sander J. D. and Joung J. K., Nat. Biotechnol., 2014, Vol. 32, No. 4, pp. 347-355). Zetsche, Gootenberg et al., Cell, In Press Corrected Proof, published online 25 Sep. 2015, describe Cpf1, a single RNA-guided endonuclease of a class 2 CRISPR-Cas system. Cpf1 was reported to mediate robust DNA interference with features distinct from Cas9. Cpf1 is a single RNA-guided endonuclease lacking tracrRNA, and it utilizes a T-rich protospacer-adjacent motif (PAM). Moreover, Cpf1 cleaves DNA via a staggered DNA double-stranded break.

There remains a need for methods and kits for retrieval of source clones from amongst a heterogeneous cell mix. In particular, a method which mitigates or avoids the disadvantage associated with the need to first sort, expand and maintain clonal populations. The present invention addresses these and other needs.

### BRIEF DESCRIPTION OF THE INVENTION

One or more of the above-described needs may be addressed by the development of a molecular “barcode reader”, which in accordance with the invention described herein makes use the ability of CRISPR to cleave selected sequences. An ability to predict the outcomes of these cleavage events in concert with a knowledge of their mutational outcome would provide a very powerful tool to identify clonal populations with phenotypic specialization within a mixture. Broadly, the present invention exploits the ability of the CRISPR-Cas9 system to target a unique predetermined DNA barcode sequence (i.e. the barcodes are used as CRISPR binding sites) in order to facilitate retrieval of source clones from amongst a heterogeneous cell mix. The present inventors have found that CRISPR-Cas9-based retrieval of cells carrying a DNA barcode of interest (the sgRNA of the CRISPR-Cas9 system targeting said barcode) permits a clonal cell population to be isolated from the heterogeneous mix for subsequent expansion and study.

Accordingly, in a first aspect the present invention provides a method for targeted cell retrieval, comprising:
providing a population of barcoded cells, said population comprising a plurality of different barcodes, each of the plurality of different barcodes being uniquely targetable with a target-specific CRISPR RNA;
introducing a CRISPR-Cas system, or one or more vectors encoding the components of the CRISPR-Cas system, into the population of barcoded cells, said CRISPR-Cas system having a target-specific CRISPR RNA that targets a first barcode of said plurality of different barcodes, thereby causing a CRISPR-Cas system-mediated change at the target site leading to a change in one or more detectable properties of at least one cell carrying said first barcode; and
retrieving said at least one cell carrying said first barcode based on the change in said one or more detectable properties.

In some cases providing the population of barcoded cells comprises transfecting, infecting or transforming a population of heterogeneous cells with a barcode library such that on average each cell is barcoded with one unique DNA barcode.

The barcoded cells may then be expanded such that each unique clone will be represented by several cells expressing the same barcode. This population can then be aliquoted into at least two samples for experimental use and for storage whereby statistically each clone will be represented in each aliquot.

In some cases the barcode library may be a viral barcode library, e.g. a retroviral or lentiviral barcode library.

In some cases the barcodes are at least 15, 16, 17, 18, 19 or 20 nucleotides in length. Preferably, the barcodes are at least 20 nucleotides in length. It is believed that CRISPR Cas9 optimally targets a sequences of 20 nucleotides in length. Longer barcodes are possible, in which case the barcode will include sequence in addition to the CRISPR target sequence. In some cases the barcodes are exactly 20 nucleotides in length.

The present inventors have found that by employing non-endogenous sequence as barcode sequence (i.e. the barcodes sequence does not match genomic sequence endogenous to the cells being targeted for retrieval), off-target CRISPR-mediated effects are minimised. Accordingly, in some cases at least 70, 80, 90, 95, 99 or 100% of the barcode sequences of said plurality of different barcodes are not endogenous genomic sequence of the cells. In some case the population of barcoded cells are of one or more taxonomic species (e.g. Homo sapiens and/or Mus musculus) and the barcode sequences of said plurality of different barcodes are not found in the endogenous genomic sequence of said one or more taxonomic species.

Additionally or alternatively, the maximum sequence identity between the barcode sequence of each barcoded cell and any endogenous genomic sequence of said barcoded cell is 70, 80, 90 or 95%, calculated over the full-length of the barcode sequence. By keeping the sequence identity between the barcodes and the cells targeted for retrieval low off-target CRISPR-mediated effects due to cross-talk are minimised. This reduces the likelihood of false-positive retrieval of cells with an unwanted barcode.

The CRISPR-Cas system may comprise an RNA-guided DNA endonuclease enzyme, which may in some cases be of Cas type II, such as CRISPR associated protein 9 (Cas9) or Cpf1.

In some cases the barcoded cells comprise a protospacer adjacent motif (PAM) sequence immediately downstream (i.e. 3′) of said barcode sequence. The PAM sequence may in some cases be of the three nucleotide sequence NGG.

In some cases the barcoded cells comprise restriction sites upstream (i.e. 5′) of the barcode sequence and/or downstream (i.e. 3′) of the barcode sequence or, where present, the PAM sequence.

In certain cases the barcoded cells comprise a selector gene that encodes a selectable marker. As will be apparent to the skilled person, a wide variety of selectable markers are known and suitable for use in the methods described herein. In particular, the selector gene may encode a fluorescent protein, an antibiotic resistance protein or a cytotoxic protein.

In some cases the selector gene is separated from said barcode sequence by a spacer sequence. The spacer sequence provides a “buffer zone” so that, e.g., CRISPR-induced deletion in the region of the barcode is less likely to result in loss of or damage to the sequence encoding the selector protein. The spacer sequence may be, for example, at least 5, 10, 15, 20 or 50 nucleotides in length. In some cases the spacer sequence lies downstream of the barcode sequence and downstream of the PAM sequence, where present, but upstream of said selector gene. In some cases the barcode is separated from the ATG start codon by said spacer sequence, which may be a non-specific sequence spacer (“NSSS”) to reduce the chances of a larger 5′ deletion event extending to the ATG translational start site.

In some cases, said barcode may be downstream of a constitutively expressed transgene. In certain embodiments one or more selector genes may be downstream of the barcode and may be placed out-of-frame (e.g. in a −1 reading frame) relative to the constitutively expressed transgene. In some embodiments a stop codon is present downstream of the barcode, but upstream of the one or more selector genes, the stop codon being in-frame with said constitutively expressed transgene. Prior to action of a barcode-targeting CRISPR-Cas system, the stop codon prevents translation of the downstream one or more selector genes. However, a CRISPR-mediated edit, e.g. a 1, 4, 7, etc, b.p. deletion brings the stop codon out-of-frame, resulting in expression of said one or more downstream selector genes. It is thought that this approach minimises the effects of multiple ATG translation initiation codons, which if present could result in 5′ truncated proteins as a result of translation initiation at internal ATGs.

In some cases, there may be provided a second barcode downstream of the first barcode, for example, downstream of the one or more selector genes. The second barcode would typically (preferably always) be different from the first barcode. In particular, the second barcode may comprise a sequencing barcode, such as a single cell sequencing barcode (e.g. a 10× Genomics single cell sequencing barcode). Optionally, a polyadenylation (polyA) sequence (e.g. bovine growth hormone polyadenylation signal) may be provided downstream, e.g., immediately downstream of the sequencing barcode. The PolyA sequence facilitates single cell sequencing of the sequencing barcode. This allows smartcodes corresponding to each single cell transcriptional profile to be ascertained.

In some cases the CRISPR-Cas system comprises:
(i) a target-specific CRISPR RNA (crRNA) and an auxiliary trans-activating crRNA (tracrRNA); or
(ii) a single guide RNA (sgRNA) comprising a fusion construct of crRNA and tracrRNA. For reasons of simplicity, the sgRNA is preferred in certain circumstances.

In some cases in accordance with this and other aspects of the present invention the selector gene is out-of-frame, and action of the CRISPR-Cas system causes the out-of-frame selector gene of the at least one cell carrying said first barcode to be brought in-frame. In particular, the CRISPR-induced reversion of the out-of-frame selector gene to an in-frame position allows the selector gene-encoded gene product to be produced thereby resulting in a detectable phenotypic change to the cell. In some cases, the action of the CRISPR-Cas system comprises addition or deletion of one or more nucleotides in or downstream of said first barcode. For example, deletion of 1, 4 or 7 nucleotides, or deletion of 2, 5 or 8 nucleotides, may be employed to bring the selector gene in-frame.

In some cases in accordance with this and other aspects of the present invention there may be more than one selector gene. In particular, there may be a first selector gene and a second selector gene, wherein the first and second selector genes are different. The second selector gene may encode a second selectable marker. In some cases the second selector gene may be out-of-frame. In particular, the second selector gene may be in the same reading frame as the first selector gene. This means that if the first selector gene is brought in-frame, for example, by CRISPR-Cas-mediated base excision or by insertion or deletion mutation (e.g. spontaneous mutation), the second selector gene will also be brought in-frame and will be expressed. The present inventors have found that, while the CRISPR-Cas system is target-specific, in certain cases there is observed a non-zero rate of spontaneous mutation that causes an out-of-frame selector gene to be brought in-frame even in the absence of or prior to CRISPR-Cas mediated base excision. In this way such spontaneous mutation gives rise to so-called “false positives”, which are cells that express the first (and second) selector genes even when they do not have the appropriate barcode to be targeted by said CRISPR-Cas system. The present inventors realised that such false positives could be minimised by employing first and second selector genes and by performing a pre-CRISPR-Cas step of selecting out those cells in which the spontaneous mutation has resulted in the first and second selector genes being brought in-frame. Accordingly, in some cases the method of this and other aspects of the present invention may further comprise a negative selection step prior to said step of introducing the CRISPR-Cas system (or said one or more vectors encoding the components of the CRISPR-Cas system), said negative selection step comprising selective removal of cells that express said second selector gene. In particular, the selective removal may comprise killing of cells based on the presence of said second selectable marker. For example, the second selector gene may encode an enzyme that confers sensitivity to a cytotoxic drug. In certain cases, the method comprises applying said cytotoxic drug to the cells prior to said step of introducing the CRISPR-Cas system (or said one or more vectors encoding the components of the CRISPR-Cas system), thereby killing at least a proportion (preferably a majority) of any cells that have said second selector gene in-frame, for example in-frame by virtue of a spontaneous mutation. As described in detail in the following Examples, the second selector gene may encode cytosine deaminase and the cytotoxic drug may be 5-fluorocytosine. Other example combinations of selector gene and selector drug include: Thymidine kinase and the drug ganciclovir (INN, USAN, BAN); gancyclovir; DHPG; 9-(1,3-dihydroxy-2-propoxymethyl)guanine, or (for non-human cells) a gene encoding the diphtheria toxin receptor (doi: 10.1074/jbc.270.3.1015, 1995, The Journal of Biological Chemistry, Vol. 270, pp. 1015-1019) and the diphtheria toxin as the selector drug. The Examples herein provide evidence that the use of a second selector gene and a pre-CRISPR-Cas negative selection step against said second selector gene improves the specificity of the subsequent CRISPR-Cas mediated target cell retrieval.

In certain cases the selector gene may be in-frame and under the control of a selector promoter. The selector promoter may be inducible or repressible by means of a transactivation domain or repressor domain, respectively. In some cases the CRISPR-Cas system comprises a Cas (e.g. Cas9) fusion protein comprising a transactivation domain or repressor domain for said selector promoter. In particular, the Cas may be a catalytically inactive endonuclease. For example, the Cas9 fusion protein may comprise a mutant Cas9 having substantially no endonuclease activity or having reduced endonuclease activity relative to wild-type Cas9. The inactive Cas9 may be directly or indirectly coupled or fused to the transactivation domain or repressor domain. The presence of, or delivery of, the matching sgRNA to the cell results in localisation of the Cas9-transactivator or Cas9-transrepressor fusion protein to the target site of the selector gene and activation or repression of the selector gene, respectively. In some cases the transactivation domain activates or induces said selector promoter. In some cases the repressor domain down-regulates said selector promoter. In particular cases the transactivation domain protein may comprise a tetracycline transactivator protein and the selector promoter may comprise a tetracycline response element (TRE). In certain cases the transrepressor protein may comprise a Kruppel associated box (KRAB) domain KRAB protein. Examples of human genes encoding KRAB domain proteins include: KOX1/ZNF10, KOX8/ZNF708, ZNF43, ZNF184, ZNF91, HPF4, HTF10 and HTF34. In certain cases the action of said CRISPR-Cas system comprises transactivation of said selector promoter thereby causing transcriptional activation of said selector gene.

In some cases the one or more vectors encoding the components of the CRISPR-Cas system comprise a Cas9-encoding gene under control of a human polymerase II promoter and/or a sgRNA-encoding gene under control of a human polymerase III promoter.

In certain cases the selector gene encodes ZS Green or Green Fluorescent Protein (GFP).

In some cases in accordance with this and other aspects of the present invention, the method comprises a preceding stage in which at least one cell from among the population of barcoded cells is selected for retrieval as a desired cell. In particular, the population of barcoded cells may be subjected to a particular environment (e.g. cell culture conditions, in vivo exposure/selection) or selection pressure (e.g. treated with a chemotherapeutic agent), which may reveal or select for a particular phenotypic property of interest (e.g. drug resistance). The at least one cell having a phenotypic property of interest may be isolated and/or obtained from the population of barcoded cells (e.g. a parallel aliquot of the population of barcoded cells stored for the purposes of retrieval) and analysed to determine the barcode that it carries. For example, the desired cell may have DNA extracted and sequenced (e.g. by next generation sequencing techniques). The identity of the barcode of the desired cell then informs the choice of CRISPR RNA (e.g. sgRNA) that is used in the cell retrieval method of the present invention so as to retrieve the desired cell having the particular phenotypic property of interest. Accordingly, in certain cases in accordance with the present invention, the method comprises a preceding step in which said first barcode is chosen for retrieval in a preceding step. The preceding step may comprise sequencing the barcode of a desired cell from said population of barcoded cells. The method may additionally comprise selecting a CRISPR RNA (e.g. sgRNA) that targets the sequence of the barcode of the desired cell, so as to retrieve the desired cell having the particular phenotype property of interest.

In some cases in accordance with the methods of the present invention, retrieving the at least one cell carrying said first barcode is carried out by making use of the change in said one or more detectable properties. In particular, the retrieval may comprise fluorescence-activated cell sorting (FACS) (e.g. where the detectable property is expression of a fluorescent protein) or culturing the cells in the presence of a selection antibiotic (e.g. where the detectable property is expression of an antibiotic resistance gene). As the skilled person will appreciate, techniques for selecting and/or isolating cells based on the detection of a selection marker are well-known in the art. All such suitable methods are contemplated herein for use with the present invention.

In some cases, the method further comprises culturing and/or expanding the at least one retrieved cell. The method may comprise storing (e.g. freezing) the retrieved cell or one or more cells descended from the retrieved cell, e.g. for subsequent study.

In some cases, the method further comprises analysing at least one structural or functional property of the at least one retrieved cell. In particular, analysing may involve a technique selected from: DNA sequencing, mass spectrometry, gel electrophoresis and gene expression profiling. In certain cases, analysis may comprise sequencing the barcode of the retrieved cell(s) to verify that the retrieved cell carries the desired barcode.

In some cases, the method further comprises subjecting the at least one retrieved cell to at least one further round of CRISPR-mediated cell selection against an independent barcode and marker. In this way the method of the invention may be carried out twice or more in series to improve the accuracy of retrieval. For example, if the retrieved cells comprises a sub-population of barcoded cells having similar (but not necessarily identical) barcode sequences, one or more rounds of further CRISPR-based cell retrieval according to the present invention using a second barcode and associated second marker may allow the retrieval of the desired cell from a sub-population of barcoded cells having similar barcode sequences. In short, second or subsequent generation cell retrieval may improve the specificity of the retrieval. However, it is specifically contemplated herein that in some cases a single round of cell retrieval may be sufficient to retrieve a cell of interest from the population of barcoded cells. As addition or alternative to a second round of CRISPR-based cell retrieval, the methods of the present invention may, in some embodiments, further comprise sequencing, e.g. single cell sequencing, of, for example, a second non-CRISPR-related barcode (a sequencing barcode), in order to verify that the desired target is sufficiently highly represented in the population for retrieval and/or subsequent study.

In a second aspect, the present invention provides a method of barcoding a population of cells so as to provide barcodes that are targetable with a target-specific CRISPR RNA (e.g. an sgRNA). The method of the second aspect may be employed to provide the population of barcoded cells for the first aspect of the invention. The method of the first aspect of the invention may comprise the method of the second aspect of the invention as the step or steps of providing the population of barcoded cells. The method of the second aspect of the invention may comprise introducing the barcodes to the population of cells so as to provide the barcoded population of cells, comprising infecting, transfecting or transforming a population of cells with a barcode library so as to provide substantially all cells with a unique DNA barcode, wherein each DNA barcode is targetable with a target-specific CRISPR RNA (e.g. sgRNA). In some cases, the cells, once barcoded, are suitable for being selectively acted on by a CRISPR-Cas system, said CRISPR-Cas system having a target-specific CRISPR RNA (e.g. sgRNA) that targets a first barcode (the “desired barcode”) of the barcodes present in the barcoded cells. In some cases the barcode library is a viral barcode library, e.g. a retroviral or lentiviral library, that is used to infect the population of cells.

In some cases the barcodes are at least 15, 16, 17, 18, 19, or 20 nucleotides in length. In certain preferred cases the barcodes are only 20 nucleotides in length.

In some cases at least 70%, 80%, 90%, 95%, 99% or 100% of the barcode sequences are not endogenous genomic sequence of the cells (i.e. the barcodes are non-naturally occurring sequence for the barcoded cells). In particular, the population of cells may be of one or more taxonomic species and the barcode sequences are not found in the endogenous genomic sequence of said one or more taxonomic species. In some cases, the maximum sequence identity between the barcode sequence of each barcoded cell and any endogenous genomic sequence of said barcoded cell is 30%, 40%, 50%, 60%, 70%, 80%, 90%, or 95% calculated over the full-length of the barcode sequence.

In some cases the barcodes introduced into the cells comprise a protospacer adjacent motif (PAM) sequence immediately downstream (i.e. 3′) of the barcode sequence.

In some cases providing the population of cells with the barcode library also comprises providing the cells with a selector gene downstream of the barcode, the selector gene encoding a selectable marker. In particular, the selector gene may encode a fluorescent protein, an antibiotic resistance protein or a cytotoxic protein.

In some cases the selector gene is separated from said barcode sequence by a spacer sequence.

In some cases the selector gene is out-of-frame.

In some cases, said barcode may be downstream of a constitutively expressed transgene. In certain embodiments one or more selector genes may be downstream of the barcode and may be placed out-of-frame (e.g. in a −1 reading frame) relative to the constitutively expressed transgene. In some embodiments a stop codon is present downstream of the barcode, but upstream of the one or more selector genes, the stop codon being in-frame with said constitutively expressed transgene. Prior to action of a barcode-targeting CRISPR-Cas system, the stop codon prevents translation of the downstream one or more selector genes. However, a CRISPR-mediated edit, e.g. a 1, 4, 7, etc, b.p. deletion brings the stop codon out-of-frame, resulting in expression of said one or more downstream selector genes which are brought in-frame by the CRISPR-mediated edit. It is thought that this approach minimises the effects of multiple ATG translation initiation codons, which if present could result in 5′ truncated proteins as a result of translation initiation at internal ATGs.

In some cases, there may be provided a second barcode downstream of the first barcode, for example, downstream of the one or more selector genes. The second barcode may be different from the first barcode. In particular, the second barcode may comprise a sequencing barcode, such as a single cell sequencing barcode (e.g. a 10× Genomics single cell sequencing barcode). Optionally, a polyadenylation (polyA) sequence (e.g. bovine growth hormone polyadenylation signal) may be provided downstream, e.g., immediately downstream of the sequencing barcode. The PolyA sequence facilitates single cell sequencing of the sequencing barcode. This allows smartcodes corresponding to each single cell transcriptional profile to be ascertained.

In some cases infecting the population of cells with the barcode library also provides the cells with at least a second selector gene downstream of the barcode, the at least second selector gene encoding a second selectable marker. The second selector gene may be out-of-frame. In particular, the second selector gene may be in the same reading frame as the first selector gene. In some cases the second selector gene encodes an enzyme that confers sensitivity to a cytotoxic drug (e.g. cytosine deaminase, which confers sensitivity to 5-fluorocytosine, Thymidine kinase and the drug ganciclovir or the gene encoding the diphtheria toxin receptor and the diphtheria toxin as the cytotoxic drug).

In some cases the selector gene is in-frame and is under the control of a selector promoter, which selector promoter is suitable for being transactivated by a transactivation domain or down-regulated by a or repressor domain and thereby being caused to alter expression of said the selector gene.

In a third aspect the present invention provides a kit for barcoding a plurality of cells and for selecting one or more cells from the barcoded plurality of cells, comprising:
a barcoding library for providing a plurality of cells substantially each with a unique barcode; and
at least one retrieval vector for providing the plurality of cells with a CRISPR-Cas system, wherein the CRISPR-Cas system comprises at least one target-specific CRISPR RNA (e.g. sgRNA) that targets at least one first barcode (“desired barcode”) of the barcodes present in the barcoding library.

In some cases the barcoding library and the retrieval vector are provided concurrently, sequentially or separately. For example, they may be provided in the form of separate containers to be used in an experiment.

In some cases the barcode library is a viral (e.g. retroviral, adenoviral or lentiviral) barcode library.

In some cases the barcodes are at least 15, 16, 17, 18, 19 or 20 nucleotides in length. In certain cases the barcodes are up to or only 20 nucleotides in length.

In some cases at least 70%, 80%, 90%, 95%, 99% or 100% of the barcode sequences are not endogenous genomic sequence of the cells intended to be barcoded. In particular, the barcode sequences may be sequences that are not found in the endogenous genomic sequence of the species of the cells intended to be barcoded. In some cases the maximum sequence identity between the barcode sequence and any endogenous genomic sequence of a cell intended to be barcoded is 30%, 40%, 50%, 60%, 70%, 80%, 90% or 95%, calculated over the full-length of the barcode sequence.

In some cases the barcodes comprise a protospacer adjacent motif (PAM) sequence immediately downstream (i.e. 3′) of the barcode sequence.

In some cases the barcoding library (e.g. barcoding library vector) also comprises a selector gene downstream of the barcode, the selector gene encoding a selectable marker. In particular, the selector gene encodes a fluorescent protein, an antibiotic resistance protein or a cytotoxic protein. In some cases the selector gene is separated from said barcode sequence by a spacer sequence.

In some cases the selector gene is out-of-frame.

In some cases, said barcode of the barcoding vector may be downstream of a constitutively expressed transgene. In certain embodiments one or more selector genes may be downstream of the barcode and may be placed out-of-frame (e.g. in a −1 reading frame) relative to the constitutively expressed transgene. In some embodiments a stop codon is present downstream of the barcode, but upstream of the one or more selector genes, the stop codon being in-frame with said constitutively expressed transgene. Prior to action of a barcode-targeting CRISPR-Cas system, the stop codon prevents translation of the downstream one or more selector genes. However, a CRISPR-mediated edit, e.g. a 1, 4, 7, etc, b.p. deletion brings the stop codon out-of-frame, resulting in expression of said one or more downstream selector genes which are brought in-frame by the CRISPR-mediated edit. It is thought that this approach minimises the effects of multiple ATG translation initiation codons, which if present could result in 5′ truncated proteins as a result of translation initiation at internal ATGs.

In some cases, there may be provided a second barcode, e.g., downstream of the first barcode, for example, downstream of the one or more selector genes. The second barcode may be different from the first barcode. In particular, the second barcode may comprise a sequencing barcode, such as a single cell sequencing barcode (e.g. a 10× Genomics single cell sequencing barcode). Optionally, a polyadenylation (polyA) sequence (e.g. bovine growth hormone polyadenylation signal) may be provided downstream, e.g., immediately downstream of the sequencing barcode. The PolyA sequence facilitates single cell sequencing of the sequencing barcode. This allows smartcodes corresponding to each single cell transcriptional profile to be ascertained.

In some cases the barcoding vector also comprises at least a second selector gene downstream of the barcode (optionally downstream of the first selector gene), the at least second selector gene encoding a second selectable marker. In some cases the second selector gene is out-of-frame. In particular, the second selector gene may be in the same reading frame as the first selector gene. In some cases the second selector gene encodes an enzyme that confers sensitivity to a cytotoxic drug (for example the second selector gene may encode cytosine deaminase, which confers sensitivity to 5-fluorocytosine, Thymidine kinase and the drug ganciclovir or the gene encoding the diphtheria toxin receptor and the diphtheria toxin as the cytotoxic drug).

In some cases the selector gene is in-frame and is under the control of a selector promoter, and wherein said CRISPR-Cas system comprises a Cas (e.g. Cas9) fusion protein comprising a transactivation domain or repressor domain for said selector promoter. In particular, the Cas9 fusion protein may comprises a mutant Cas9 having substantially no endonuclease activity or having reduced endonuclease activity relative to wild-type Cas9.

Non-limiting examples of utility of the retrieval system of the present invention.

The invention in its various aspects described herein will have utility in a wide range of contexts including retrieval of desired clones following application of a selection pressure to an experimental cell sample intended to select or identify a desired phenotype. Such a use could be useful in a variety of fields including; i) in biotechnology to retrieve desired clones after experimental selection of cells designed to produce products such as recombinant proteins or other materials, ii) retrieval of resistant clones following experimental exposure to cytotoxic agents such as drugs, iii) retrieval of clones after in vivo selection for a desired phenotypic property which could include in oncology to identify cells with properties such as metastatic ability, engraftment ability, survival in a host, iv) retrieval of clones following labelling of stem cell or progenitor cell populations and selection or isolation of cell types with a desired phenotype such as development lineage ability, cell type generation etc., v) in vivo selection of cells in a host to allow retrieval of clones with a therapeutic property such as ability to form a cell type of interest, ability to express a therapeutic substance in vivo, ability to locate to an area of interest, ability to engraft in a host, ability to replenish/replace a host tissue/cell type etc. Moreover, the present inventors contemplate use of the invention in the retrieval of T-cells or other immune cells which recognize specific epitopes.

In a fourth aspect, the present invention provides a method for creating an artificial CRISPR target site at a genomic site of a cell, the method comprising introducing a CRISPR target sequence and protospacer adjacent motif (PAM) site into the genome of the target cell, wherein the CRISPR target sequence is a sequence which, prior to its introduction, is not found in the endogenous genomic DNA sequence of the target cell. In some cases, the target cell is a mammalian or human cell or bacterial or insect cell.

In a fifth aspect, the present invention provides a method for altering or controlling expression of a target gene, said method comprising:
providing a cell having an artificial CRISPR target site, the sequence of which is exogenous to the genome of the cell, wherein said artificial CRISPR target site is upstream of the target gene; and
introducing a CRISPR-Cas system, or a vector encoding the components of the CRISPR-Cas system, into the cell, wherein the CRISPR-Cas system comprises a target-specific CRISPR RNA (e.g. an sgRNA) that targets said artificial CRISPR target site, and wherein the said CRISPR-Cas system causes up-regulation or down-regulation of expression of the target gene. In some cases the target gene is exogenous to the cell.

In some cases the target gene is out-of-frame and the CRISPR-Cas system causes the target gene to be brought in-frame.

In some cases the target gene is in-frame and the CRISPR-Cas system comprises a transactivation or repressor domain that acts on the promoter of the target gene to up-regulate or down-regulate expression of the target gene.

The present invention in its various aspects may be put to a wide variety of uses. In relation to the control of genes, such uses may for example include: i) labelling a population of cells intended for use in a cell therapy with a barcode corresponding to a CRISPR targeting RNA linked to a selector gene to enable manipulation of the cells to turn on or off the selector gene to regulate the activity or phenotype of the cells. For example, use of an out-of frame cytotoxic selection marker in a cell therapy would enable the cells to be killed by exposure to the appropriately matched CRISPR RNA vector to revert the marker gene into frame. Conceptually any gene could be regulated in this manner to either place it back into frame and express the gene or through use of the transactivation or transrepression systems described herein to increase or decrease the expression of a selector gene. The selector gene could be a selectable marker or could itself be a gene with therapeutically beneficial effects but whose expression needs to be controlled. The CRISPR targeting component could be contained within the cell therapy prior to administration or delivered at a later point to the patient. The CRISPR systems could themselves be regulated by an inducible promoter system responsive to an external stimulus (such as tetracyclin or similar) such that the CRISPR event could be controlled by delivery of an inducer rather than delivery of the CRIPSR system itself to the cell therapy cells.

Example of cell based therapies could include immune cell therapies such as chimeric antigen receptor T cells where mechanisms to regulate or switch off the T cell function could be useful for managing their activity and potential side effects. Other examples could include stem cell transplantation, cellular transplantation of cells to produce therapeutic proteins within the host (e.g. pancreatic cells to produce insulin).

The present invention includes the combination of the aspects and preferred features described except where such a combination is clearly impermissible or is stated to be expressly avoided. These and further aspects and embodiments of the invention are described in further detail below and with reference to the accompanying examples and figures.

### BRIEF DESCRIPTION OF THE FIGURES

FIG. 1 shows a schematic representation of a DNA barcode experimental workflow. A. A library of retroviral vectors containing unique DNA barcode identifiers is synthesised. B. A population of heterogeneous cells is infected to incorporate barcodes into genome. C & D. A heterogeneous cell population is introduced into an in vivo system (e.g. tumour implants in a mouse) and a particular cell population is isolated from the in vivo system based on an in vivo property (e.g. drug resistance). E. Next-generation sequencing of DNA from selected cells is carried out. F. The sequences of individual barcode identifiers of the isolated cell DNA are determined.

FIG. 2 shows a schematic representation of the experimental workflow of CRISPR-mediated retrieval of a barcoded clonal cell population from a heterogeneous cell population. A. A population of heterogeneous cells is infected with a retroviral barcode library and the library is split into fractions which based on the distribution should result in each fraction containing a representative cell for each specific barcode. B. The heterogeneous cell population is introduced into an in vivo system to select for cells with desired in vivo properties. C. Next-generation sequencing of DNA from selected cells is performed. D. The sequence of individual barcode identifiers in the isolated cell DNA is determined. E. CRISPR-Cas9 is introduced via retroviral infection into a stored aliquot of the barcoded heterogeneous cell population, with gDNA complementary to the identified barcode sequence. CRISPR-Cas9 cleavage of the barcode places a selector gene (e.g. fluorescence or antibiotic resistance) in frame, or alternatively CRISPR-Cas9 transcriptional activation of an in-frame selector under the control of a synthetic promoter, allows single cells or a single clonal cell population to be selected and expanded.

FIG. 3 shows a schematic representation of a construct comprising the dual-function barcode/CRISPR target site and selector. NSSS is a non-specific spacer sequence, RS is a restriction site (to facilitate addition of the barcode library), CrispR Barcode/gRNA binding site is the 20 bp sequence that acts both as a DNA barcode and a CRISPR target site that is bound by its corresponding gRNA, PS is a protospacer adjacent motif (PAM) sequence, streptavidin binding spacer is a mutated gene sequence having start and stop codons removed the purpose of which is to act as a spacer between the CRISPR target site and the downstream selector, and ZS Green is an example of a selector gene (other examples include different florescent proteins, antibiotic resistance gene or a destructive protein e.g. the diphtheria toxin) which is initially out-of-frame, but which falls into frame upon action of CRISPR-Cas9 at the CRISPR target site (e.g. excision of 1, 4 or 7 nucleotides in the barcode/CRISPR target sequence).

FIG. 4 shows MacsQuant® (flow cytometry) plots. A & B: show example forward and side scatter plots; C: DGCR8 SMARTCODE no cas9/gRNA; D: GFP SMARTCODE no cas9/gRNA; E; DGCR8 SMARTCODE+DGCR8 cas9/gRNA; F: DGCR8 SMARTCODE+GFP cas9/gRNA; G: GFP SMARTCODE+DGCR8 cas9/gRNA; and H: GFP SMARTCODE+GFP cas9/gRNA.

FIG. 5 shows a schematic representation of a construct comprising a modified dual-function barcode/CRISPR target site having both a positive selector and a negative selector. ATG is the translation initiation codon. NSSS is a non-specific spacer sequence, RS is a restriction site (to facilitate addition of the barcode library), CrispR Barcode/gRNA binding site is the 20 bp sequence that acts both as a DNA barcode and a CRISPR target site that is bound by its corresponding gRNA, PS is a protospacer adjacent motif (PAM) sequence, Puro R is a puromycin resistance gene and is an example of a positive selector gene which is initially out-of-frame, but which falls into frame upon action of CRISPR-Cas9 at the CRISPR target site (e.g. excision of 1, 4 or 7 nucleotides in the barcode/CRISPR target sequence), and CodA is a cytosine deaminase gene, which when in-frame renders cells sensitive to the toxic effects of 5-fluorocytosine and is therefore an example of a negative selector gene. In this example Puro R and CodA are in the same frame (initially out-of-frame, but are brought in-frame by CRISPR-Cas9-induced excision of 1/4/7 nucleotides in the barcode/CRISPR target sequence).

FIG. 6 shows a bar graph of % (y-axis) of total sequencing reads having a frame-shift mutation in the smartcode region that puts the puromycin resistance gene in-frame. The left-most bar (“FC 500 Puro”) shows cells treated with 5-fluorocytosine having a 1:500 ratio of Pasha:GFP barcodes (i.e. P(G)) after CRISPR/Cas9 treatment and puromycin treatment. The second bar moving right (“no puro”) shows cells treated with 5-fluorocytosine having a 1:500 ratio of Pasha:GFP barcodes (i.e. P(G)) after CRISPR/Cas9 treatment but without puromycin treatment. The third bar moving right (“FC 1000 Puro”) shows cells treated with 5-fluorocytosine having a 1:1000 ratio of Pasha:GFP barcodes (i.e. P(G)) after CRISPR/Cas9 treatment and puromycin treatment. The fourth bar moving right (“no puro”) shows cells treated with 5-fluorocytosine having a 1:1000 ratio of Pasha:GFP barcodes (i.e. P(G)) after CRISPR/Cas9 treatment but without puromycin treatment. The fifth bar moving right (“FC 10000 Puro”) shows cells treated with 5-fluorocytosine having a 1:10000 ratio of Pasha:GFP barcodes (i.e. P(G)) after CRISPR/Cas9 treatment and puromycin treatment. The right-hand most bar moving (“no puro”) shows cells treated with 5-fluorocytosine having a 1:10000 ratio of Pasha:GFP barcodes (i.e. P(G)) after CRISPR/Cas9 treatment but without puromycin treatment.

FIG. 7 shows an alternative arrangement (“Smartcode strategy 2”). A smartcode is placed downstream of a constitutively expressed transgene (Transcript 1). One or more transcripts (Transcript 2 and 3) are also placed downstream of the smartcode, in −1 frame. These can be activated when a 1, 4, 7 . . . etc. base pair deletion is introduced by targeting the smartcode with CRISPR/Cas9. A second barcode can also be inserted downstream of the Cas9 activated transcripts, where a poly-adenylation signal (e.g. bovine growth hormone). Placement of this second barcode next to the poly-adenlyation site allows for the capture of the barcode sequence using single cell sequencing technologies (e.g. 10× Genomics). The upper portion of the Figure shows the open reading frame (ORF) prior to CRISPR/Cas9 treatment, in which the stop codon is in-frame and upstream of the Transcript 2. The lower portion of the Figure shows the ORF after CRISPR/Cas9-induced deletion of, e.g., 1, 4, 7, etc. nucleotides. The Stop codon is no longer in-frame and the transcript 2 and transcript 3 genes are brought in-frame and are expressed. A second barcode (BC) is shown downstream of the smartcode CRISPR-targeted barcode.

FIG. 8 shows fluorescence microscopy images in which ZsGreen and mCherry expression levels are visible in different mixtures of BC.A and BC.B infected cells after transfection with Cas9 and either BC.A or BC.B. The left-most panel shows sgRNA-BC.A targeted BC.B mCherry red fluorescent protein (RFP) expression after Cas9 targeting. The next panel to the right shows sgRNA-BC.B targeted BC.B RFP expression after Cas9 targeting. The middle panel shows sgRNA-BC.B targeted BC.A+BC.B 1:1 mix RFP expression after Cas9 targeting. The next panel to the right shows sgRNA-BC.B targeted BC.A+BC.B 100:1 mix RFP expression after Cas9 targeting. The right-most panel shows BC.A+BC.B 1:1 mix ZsGreen expression.

FIG. 9 shows Sanger sequencing .abl traces of the PCR amplified smartcode region from mixtures of BC.A and BC.B infected cell populations, both before and after cells were transfected with Cas9 and a BC.B targeting sgRNA, and mCherry positive cells were isolated using FACS.

FIG. 10 shows single cell RNA sequencing data from 4T1 breast cancer cells, which had been infected with a complex barcode library allowing the barcode to be captured in the single cell sequencing data. A) Shows 11 tSNE clusters; B) shows the clusters of A after the SCseq barcode identities were overlaid. It is apparent that the individual tSNE clusters represent distinct barcoded populations.

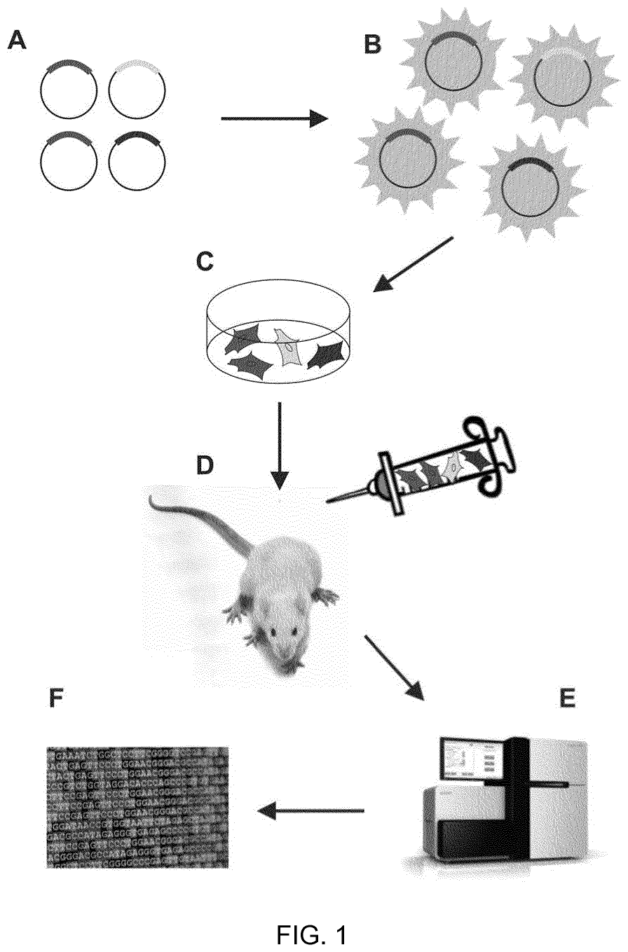

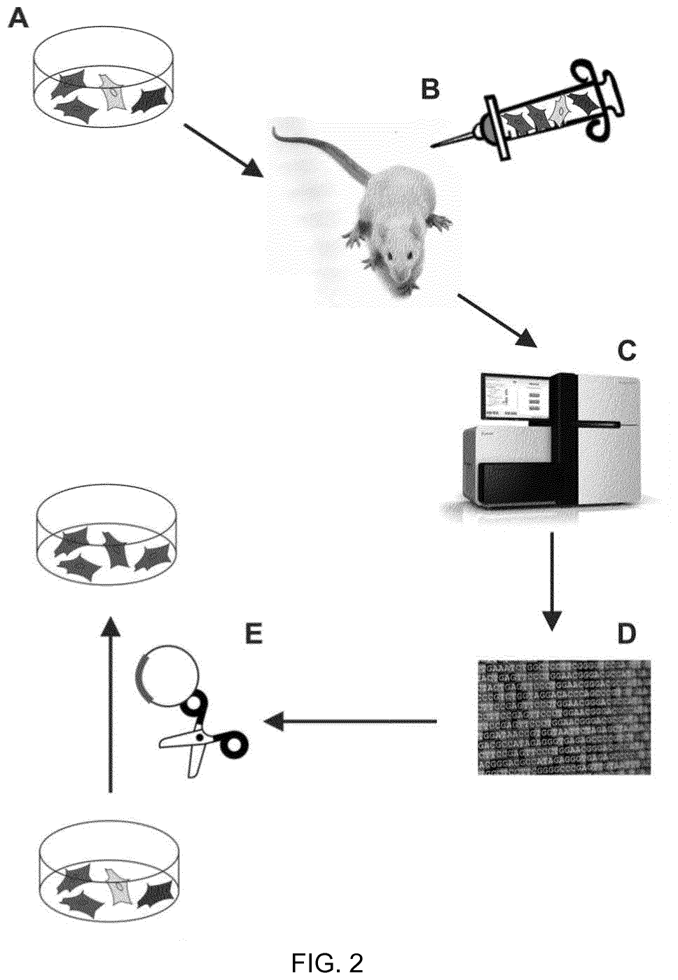

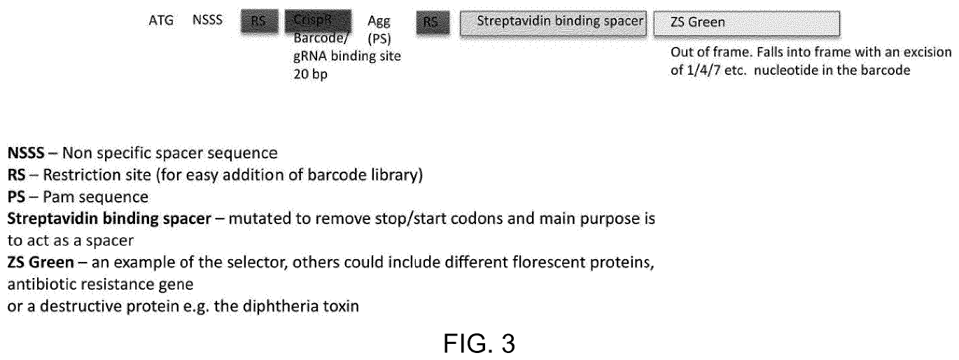

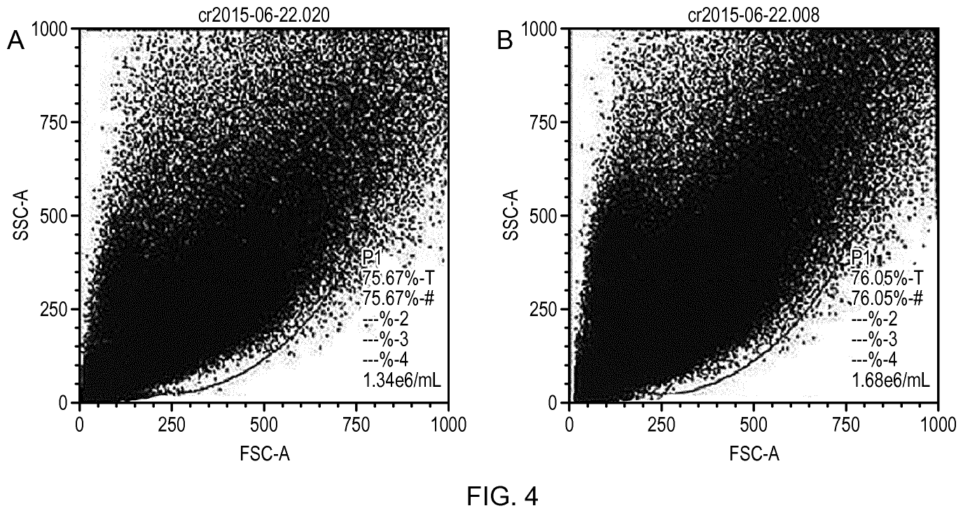

### DETAILED DESCRIPTION OF THE INVENTION

Aspects and embodiments of the present invention will now be discussed with reference to the accompanying figures. Further aspects and embodiments will be apparent to those skilled in the art. All documents mentioned in this text are incorporated herein by reference.

In describing the present invention, the following terms will be employed, and are intended to be defined as indicated below.

“CRISPR” is an abbreviation of “clustered regularly interspaced short palindromic repeats”. As used herein CRISPR or CRISPR/Cas system means a targeted gene/DNA editing system, typically having a RNA-guided DNA endonuclease effector (such as Cas9) and a CRISPR RNA that guides the effector (e.g. a single guide RNA or sgRNA). The CRISPR/Cas system may be active or catalytically inactive. In the latter case, the inactive Cas may be fused with or coupled to a transactivation domain or repressor domain for regulating a promoter and thereby regulating expression of a gene. Specifically contemplated herein are suitable CRISPR/Cas systems, such as Class II Cas genes Cas9 and Cpf1.

“Barcode” means a nucleotide sequence (e.g. DNA) that may be used to uniquely tag or label a cell among a population of cells. The barcode may be read by sequencing the DNA of the cell to identify which barcode the cell carries. The barcode may in some cases be integrated into the genome of the cell or may be extra-chromosomal.

As used herein a barcode may comprise a CRISPR sgRNA target site and may be referred to herein as a “smartcode”.

“Selector gene” (also known as a reporter gene) means a gene that encodes a gene product that confers on the organism expressing it a characteristic that is easily identified, measured or revealed (e.g. under pre-defined conditions such as upon exposure to a particular chemical). Selector genes could be positive selection for the desired marker or negative selection of those cells lacking the desired marker. Many examples of such marker genes are well-known in the art and include, for example, fluorescent proteins, enzymes with detectable products, cell surface proteins detectable by various methods including FACS or magnetic bead sorting, antibiotic resistance genes, genes with cytotoxic effects, beta-galactosidase, chloramphenicol acetyltransferase, green fluorescent protein and red fluorescent protein. The selector gene may give rise to a qualitatively or quantitatively detectable property that distinguishes cells expressing the selector gene from those not expressing the selector gene or expressing the selector gene at a lower level. The detectable property may be detectable directly (e.g. with appropriate imaging or measuring apparatus) or indirectly (e.g. following development or exposure to particular conditions). It is immaterial whether the selector gene is switched on against a background of non-expressing cells or switched off against a background of expressing cells.

In comparing CRISPR and short hairpin RNA (shRNA) screens, each have unique advantages and disadvantages. shRNAs can knock down expression of target genes by 90% on a population level but the degree of inhibition in individual cells varies. This leads to variation in phenotypes, which can confound the analysis of screens on a genome-wide scale. In contrast, if CRISPR-mediated editing results in a null phenotype, the outcome is very uniform, even though the event may not occur in the majority of the population.

In one embodiment of the invention, we use a barcode that is targetable with a target-specific CRISPR RNA (“smart code”), appended in cis to one or more sgRNA expression cassettes designed to target endogenous genes, to increase the likelihood that in a given cell, editing has occurred. We observed in our analysis of the smart code vectors that upon selection for editing of the selectable marker, a co-selected gene (GFP) integrated in an independent genomic locus was edited with extremely high efficiency. This correlates with an observation made using our dual-sgRNA libraries, wherein deletions predominated over single site mutations. We interpret that to mean that in individual cells where editing occurs, it is more common to cut both sites, and thus recombine, than it is to cut one site, and thus mutate them individually.

Considering these two unexpected observations together, without wishing to be bound by any particular theory, the present inventors conclude that efficient editing may be a cell autonomous phenotype. Cells which edit one locus are more likely to edit another. We propose that this principle can be used to construct highly efficient sgRNA libraries. By incorporating into a single construct a guide sequence targeting a genomic region of interest and a guide targeting a marker encoded in cis on the same vector, we can use the cis-linked marker to enrich for cells in which genomic editing has occurred. In certain cases, the guide sequence targeting the genomic region of interest and the guide sequence targeting the marker encoded in cis on the same vector are the same sequence. In certain cases, the guide sequence targeting the genomic region of interest and the guide sequence targeting the marker encoded in cis on the same vector are the different sequences.

In a simple example, a vector encoding an sgRNA targeting an endogenous locus also contains an sgRNA able to active, by inducing a frame shift, a selectable marker. In one embodiment, that marker is a drug selection that is placed in frame and becomes functional upon editing. Selection for cells that have edited the marker will enrich for cells that have edited the endogenous gene.

Applying this concept on a single gene or genome wide scale has the potential to optimize the potential of gene editing.

The following is presented by way of example and is not to be construed as a limitation to the scope of the claims.

The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilised for realising the invention in diverse forms thereof.

While the invention has been described in conjunction with the exemplary embodiments described above, many equivalent modifications and variations will be apparent to those skilled in the art when given this disclosure. Accordingly, the exemplary embodiments of the invention set forth above are considered to be illustrative and not limiting. Various changes to the described embodiments may be made without departing from the spirit and scope of the invention. For the avoidance of any doubt, any theoretical explanations provided herein are provided for the purposes of improving the understanding of a reader. The inventors do not wish to be bound by any of these theoretical explanations.

Any section headings used herein are for organizational purposes only and are not to be construed as limiting the subject matter described.

Throughout this specification, including the claims which follow, unless the context requires otherwise, the word “comprise” and “include”, and variations such as “comprises”, “comprising”, and “including” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment. The term “about” in relation to a numerical value is optional and means for example +/−10%.

### EXAMPLES

### Example 1—CRISPR-Mediated Reversion of Fluorescence and Cell Sorting

Two CRISPR targets, one for GFP and the other for DGCR8, have been investigated for CRISPR-mediated reversion of fluorescent protein expression. Hek293 cells were infected with a retrovirus expressing mCherry fluorescent protein and a barcode linked to either GFP or DGCR8 out of frame reporter genes. These cells were expanded and then infected with a Cas9/gRNA lentivirus, targeting either GFP or DGCR8 linked barcodes.

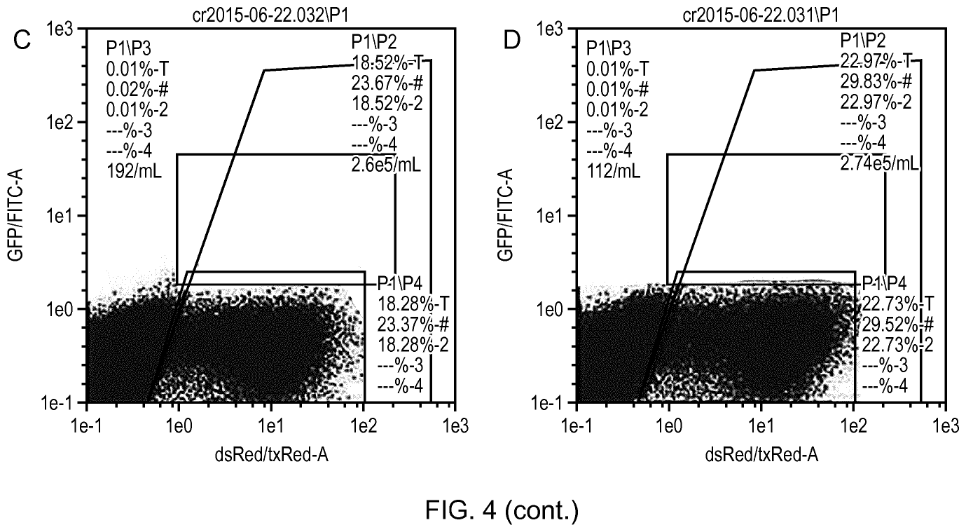

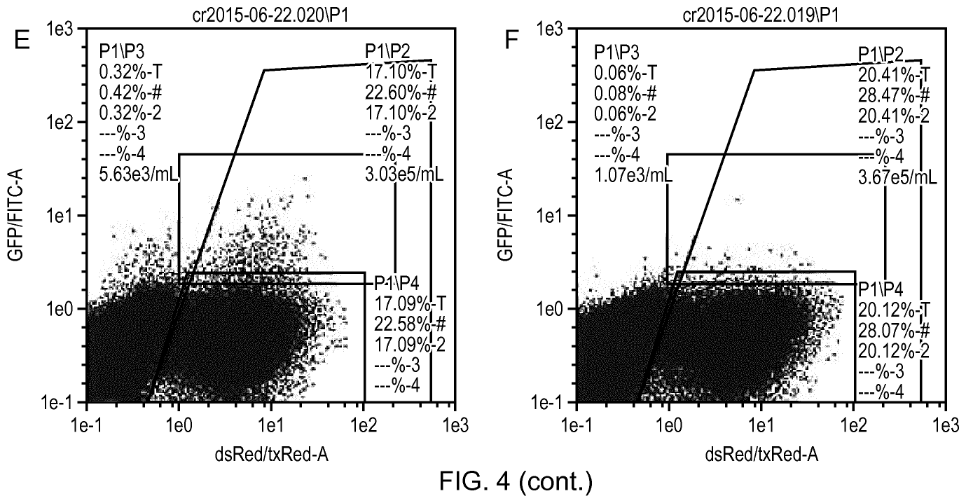

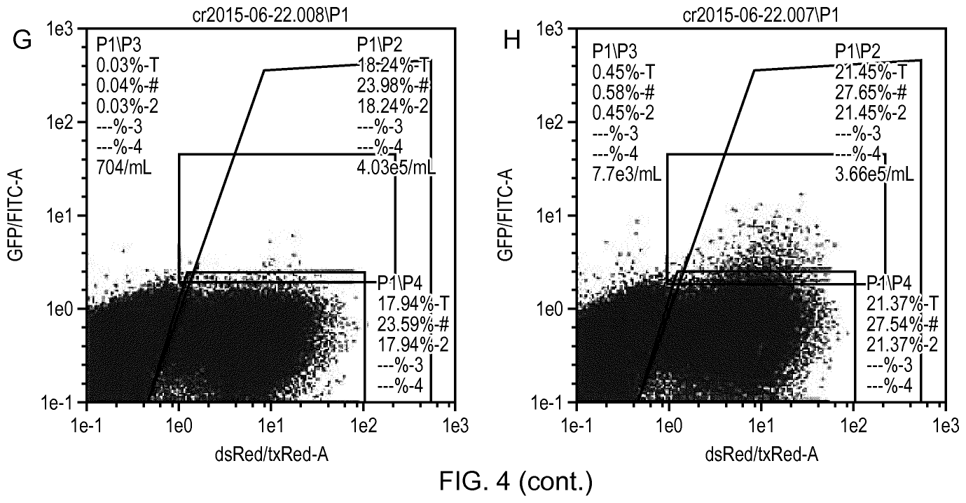

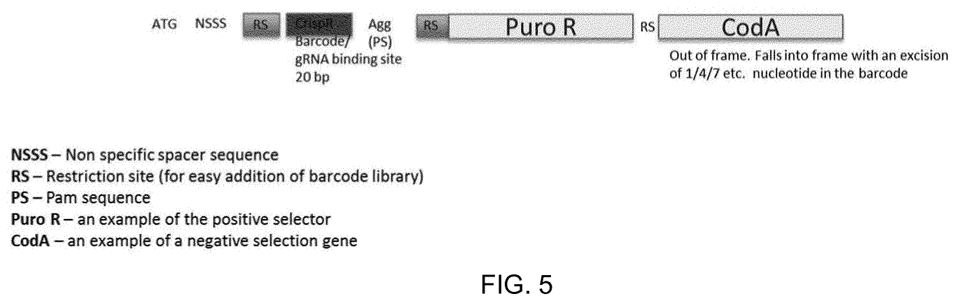

72 hours later after infection with the relevantCas9/gRNA expressing retrovirus, the infected cells were analysed by flow cytometry using a MacsQuant apparatus (see FIG. 4).

The results show an increase in GFP positive cells in panels E (DGCR8 linked barcode+DGCR8 cas9/gRNA) and H (GFP linked barcode+GFP cas9/gRNA), indicating successful CRISPR-mediated reversion of the reporter gene into frame and expression of the reporter gene.

In addition the results show the speed of the Cas9/gRNA to act on its target and that it has a high degree of specificity. A slight (but not statistically significant) increase in zsgreen positive cells was seen when measured at a later time point. Without wishing to be bound by any particular theory, the inventors presently believe that cas9/gRNA retrovirus infection was approximately 30% efficient, and the resulting nucleotide changes after CRISPR mediated cleavage and repair could place the ZSgreen sequence into frame only ⅓ of the time. Without wishing to be bound by any particular theory, the inventors presently believe that following further optimization of infection, and using deletion predictive barcodes, substantially higher positive signal is anticipated.

FACS sorting of the retrieved positive clones would enable their downstream expansion to provide a source of the desired cells matching the cell clone identified from its barcode following experimental selection. Retrieved clones can easily be verified by sequencing of the target site to confirm that the retrieved clone matches the selected clone's barcode.

These results appear to show that the percentage of cells that are non-specifically activated is very low. Further experiments are contemplated in which fluorescence-activated cell sorting (FACS) will be employed to sort these cells and sequence the barcode within, to establish if they have truly been targeted in the same manner as the specifically activated cells, or if they are simply background florescence. In addition, the plots show that some cells that are not expressing mcherry are capable of expressing zsgreen in a targeted fashion. This could be a result of the different promoters being used for each fluorescent protein (this is also seen when the barcode is designed to mimic a CRISPR reaction—data not shown). Further optimization is contemplated.

### Prophetic Example 2—CRISPR-Mediated Reversion of GFP and Retrieval of Selected Pancreatic Clonal Cells from a Heterogenous Pancreatic Cancer Cell Line

1,000,000 20-mer DNA sequences were selected randomly from the 4\hat{ }20 possible types and filtered using:
1. Positively selected for basic compatibility to CRISPR gRNA using bespoke algorithms based on machine learning;
2. Negatively selected those sequences having high homology to the human genome; and
3. Positively selected if there is a reasonable level of confidence in the size of the resulting deletion would be compatible for GFP reversion.

This resulted in an initial selection of 250 barcode sequences.

A library of barcode sequences will be used to infect a pancreatic cell line. Using the vector system described in Example 1, the inventors will use CRISPR mediated reversion of an out-of frame reporter gene to enable retrieval of several different clones from amongst the heterogeneous cell mix based on their individual barcode sequence using CRISPR to revert the out of frame GFP reporter into frame permitting GFP expression and subsequent detection by fluorescence of cells with the desired matching barcode.

It is contemplated that this prophetic example could be performed on a larger scale with thousands, tens of thousands, or hundreds of thousands of barcodes/labelled cells.

### Prophetic Example 3—CRISPR-Mediated Retrieval of Drug-Resistant Clones

As a first step CRISPR binding site barcodes will be designed. Initially, two published CRISPR target sites that have 20-30% efficiency for CRISPR-chromosomal rearrangements will be tested. Next a retrovirus barcode library will be created and used to infect a cell line in order to insert the barcodes. Initially, the experiment will test a pancreatic cell line of a known heterogeneity, where gemcitabine resistant clones have a DCK mutation, and also a mouse breast cancer cell line (4T1) where resistance to doxorubicin is correlated with increased P-glycoprotein.

Next the cells will be expanded and divided into aliquots.

Cells will then be transplanted into mice and aliquots stored in the freezer.

The mice carrying the transplanted tumour cell lines will be treated with Gemcitabine (pancreatic) or Doxorubicin (4T1).

It is thought that resistant clones will survive and colonize the cancer.

The surviving clones will be sequenced (e.g. using next generation DNA sequencing) to identify the barcode sequence.

A frozen aliquot of barcoded cells will be recovered from the library.

The barcoded cells will be treated with a CRISPR against the identified barcode. As a control, a second group of cells will be treated with a CRISPR that targets a different barcode in a separate dish.

It is anticipated that CRISPR will create a frame shift allowing, for example, the fluorescent protein ZSgreen to be put back into frame and be expressed.

The cells will then be subjected to FACs sorting (or treated with drug selection). Those cells that turn the fluorescent protein on (or culture all cells that are resistant to the drug) are thereby recovered.

Finally, the recovered cells will be sequenced. It is anticipated that the ZSgreen positive cells will have the DCK mutation or increased P-glycoprotein required for survival in presence of Gemcitabine or Doxorubicin, respectively.

### Example 4—Improved Targeted Cell Retrieval

The inventors had observed some spontaneous mutations whereby deletions of 1, 4, or 7 base pairs led to putting Puro back in-frame meaning these cells get selected by puromycin even in the absence of any CRISPR step. In order to overcome this problem and reduce the “false positive” rate of retrieval in the absence of the correct CRISPR target barcode, the inventors decided to employ an additional (negative) selector, which could be used to screen out any spontaneously mutated cells prior to the CRISPR/Cas9-effective excision.

In this example, a negative selection was used to reduce the background “false positive” rate. This is exemplified by employing Puro R (puromycin resistance gene) as a positive selector and CodA (cytosine deaminase) as a negative selector. The vector comprising the Puro R and CodA has both genes out-of-frame for being translated. They are however in the same frame as each other. If a spontaneous excision mutation happens in the Puro R it would also cause the CodA to move back in-frame and be translated. As such, selection with the drug 5-fluorocytosine (5-FC) would kill these cells and doing this before any CRISPR event removes these false positives.

The method then continues with the CRISPR retrieval step whereby the CRISPR event causes the 1, 4 or 7 bp excision to put the Puro R in-frame to enable puromycin-based selection for those cells that have the correct barcode/CRISPR selection event. By minimizing the false positive error rate, target-specific retrieval is improved accordingly.

Methods

The sequence for ecodeD314A (Cytosine deaminase) was cloned to the 5′ end (in-frame) of the Puro R sequence. This was done to reduce the background puromycin-resistant mutants, where mutations arose in the virus production which left a cell resistant to puromycin without CRISPR/Cas9 treatment. Cells were then treated with 5-fluorocytosine which kills cells expressing cytosine deaminase.

There is a neighbor effect with this treatment but when the cell population expressing cytosine deaminase is low this effect is minimal.

Prior experiments were done and 90 μg/ml was found to be the optimal concentration for killing the particular cells expressing CodA employed in the present experiment without affecting non-CodA-expressing cells (i.e. minimizing the aforementioned neighbor effect).

Experimental Layout

293 cells were infected with a viral plasmid containing the smartcode for either a Pasha target sequence (P) or a GFP target sequence (G). The viral plasmid also has a constitutive GFP gene expressed.

Cells were then FACs sorted based on positive fluorescence to create a stock of 293 cells ˜85% positive for fluorescence.

The Stock cell populations were then separated to “no FC treatment” (NFC) and “FC treatment” (FC).

The FC treated cells were given 90 μg/ml of 5-fluorocytosine for 3 days, washed and allowed to recover for a further 4 days.

Dilutions were then set up under the following conditions for all treatments with half a million cells majority cells plated in a 10 cm dish.

1:500

1:1000

1:10,000

G(P)—where P is the minority and G is the majority.

P(G)—where G is the minority and P is the majority.

Each condition was then infected with a viral plasmid containing Cas9 and the guide targeting the minority barcode, e.g., for 1:500 P(G), where there are 500 times more pasha barcode infected cells than there are GFP infected cells, the plate is infected with the GFP guide.

The CRISPR/Cas9 was system was given 7 days to target cells. (Based on previous data, 11 days provides the most mutations but it is a progressional system where 7 days is sufficient.) Cells were split ⅕ when confluent during this time to reduce the risk of losing the minority cells.

After CRISPR treatment the cells were treated with puromycin. The few cells remaining after puromycin treatment were resistant to puromycin and allowed to expand.

Macsquant analysis of the GFP positive cells was carried out immediately prior to puromycin treatment and after puromycin treatment.

DNA was collected from the puromycin resistant cells for subsequent sequencing analysis (described further below).

Results and Data Analysis

1. Macsquant Data of GFP Positive Cells:

P represents cells infected with the Pasha target sequence.

G represents cells infected with the GFP target sequence.

FC is treatment with 5-fluorocytosine on both P and G prior to the dilutions set up (FC1=3 FC days treatment).

### Examples

1:1000 P(G) is 1 cell with GFP target and 999 cells with Pasha target.

1:10,000 G(P) is 1 cell with Pasha target and 9999 cells with GFP target.

GFP Positive Cells Pre-CRISPR/Cas9, Pre-Puromycin

GFP—84.5%

Pasha—85%

GFP FC1—81.5%

Pasha FC1—81.2%

GFP Positive Cells Post-CRISPR/Cas9, Post-Puromycin

TABLE 1
GFP positive cells post-CRISPR/Cas9, post-puromycin

| dilution | P(G) | G(P) | FC P(G) | FC G(P) |
|---|---|---|---|---|
| 1:500 | | | 1.3% | 95.9% |
| 1:1000 | 6.3% | 88.5% | 4.5% | 96.8% |
| 1:10,000 | 10.6% | 92.6% | 4.2% | 89.2% |

TABLE 2
Approximate cell colony coverage, and cell counts per 100 μ1

| dilution | P(G) | G(P) | FC P(G) | FC G(P) |
|---|---|---|---|---|
| 1:500 | | | ~40/6604 | ~80/22326 |
| 1:1000 | ~90/38827 | ~40/5426 | ~50/12308 | ~50/28262 |
| 1:10,000 | ~50/22995 | ~20/4017 | ~50/17090 | ~30/5070 |

Discussion and Explanation of Results
The Minority cell population was targeted with the corresponding CRISPR/Cas9 guide. For example, P(G) was targeted with the GFP guide.
All cells that were targeted with the GFP guide and where CRISPR/Cas9 has worked effectively will have the GFP signal depleted (regardless of whether the cell has a P or G target sequence), as the fluorescent protein (GFP) is read from a different reading frame. This was apparent with a post-CRISPR/Cas9 and pre-puromycin reading of FC1 P(G) 1:1000 having 3% GFP positive cells.
Cells treated with FC should have a lowered background (random mutation pushing the Puro R into frame) effect. This can be seen clearly by comparing FC columns with the corresponding non-FC treated column (e.g. 1:1000 FC P(G)=4.5% vs. P(G)=6.3%).
The P(G) dilutions are expected to have increasing % of GFP positive cells as the dilutions increase. This is under the principle that background cells will be randomly mutated to be Puro R positive, yet have not had the CRISPR work effectively, either on the target sequence or the GFP fluorescent protein sequence. As the “true” population decreases in number (with increasing dilutions), then the background population becomes more greatly represented. This can be seen in both test conditions, e.g. FC1 P(G) 1:500-1.3% and FC 1 P(G) 1:10,000-4%.
Dilutions with CRISPR/Cas9 targeting the P cells see an enrichment of GFP positive cells, e.g. the Pasha cells initially were 80-85% GFP positive pre-puromycin and after puromycin treatment this increased to ˜90-100%.
The lowered selection of cells within the 1:10,000 dilutions is likely a result of infected cells being lost as the plates were passaged over time.
Cell colony coverage (Table 2) is an estimate. It should be noted that the background cells expand with greater efficiency as they have not been exposed to any active CRISPR/Cas9.

2. Myseq (Sequencing) Data of the Barcode Region in the Cell Population Post-Puromycin Treatment.

TABLE 3
Numbers represent % (of the total reads) that have a frame shift
mutation in the smartcode region that will put Puro R inframe.

| | | | | | No cas | No cas |
|---|---|---|---|---|---|---|
| dilution | P(G) | G(P) | FC P(G) | FC G(P) | FC P(G) | FC G(P) |
| 1:500 | | | 0.37 | 54.2 | 0.15 | 0.49 |
| 1:1000 | 0.15 | 19.5 | 0.26 | 51 | | |
| 1:10,000 | 0.15 | 1.8 | 0.27 | 30.8 | | |

TABLE 4
Numbers represent % (of the total reads) that have
the targeted smartcode in the cell population.

| | | | | | No cas | No cas |
|---|---|---|---|---|---|---|
| dilution | P(G) | G(P) | FC P(G) | FC G(P) | FC P(G) | FC G(P) |
| 1:500 | | | 0.5 | 87 | 0.1 | 4.2 |
| 1:1000 | 0.8 | 29.5 | 0.4 | 72 | | |
| 1:10000 | 0.1 | 2.5 | 0.4 | 44 | | |

Discussion of Sequencing Results

From the above data it is clear that with G as a majority and thus targeting P, we get a substantial enrichment for the number of cells containing the P smartcode (Table 4), and most of these are a result of a frameshift that pushes Puro into frame in that region (Table 3). Even with a 1:10,000 dilution, so if looking for an incredibly rare cell, you still get a substantial enrichment. This is expected to enable retrieval of a target cell from a heterogeneous population even when the target cell is present at very low levels (e.g. 1 in 10,000 cells).

These numbers go up for targeting P when you look at the % of reads that contain the targeting sequence, regardless of the targeted frameshift. This is possibly due to a more downstream ATG being pushed into frame and puro still being expressed.

In addition, one must take into account intrinsic PCR and sequencing error that may result in a variable appearance of mutations in the smartcode area. For the Pasha smartcode the most frequently observed mutation was a deletion of 1 or 4 bases.

In a more complex setting, with many different cell types within the population, the remaining % of cells that were not targeted would contain a mixture of signals, so the targeted cell type would have an overwhelming signal compared to a non-targeted population.

Without wishing to be bound by any particular theory, the present inventors believe that the GFP smartcode was targeted and at the same time the corresponding sequence within the GFP fluorescent protein gene was also targeted. It is possible that when these two regions were targeted they actually removed the entire length of DNA between the two points. Previous work indicates that with two target sites the most common mutation is deletion between the two sites.

This being the case, the whole region between the two target sites would be deleted, which would remove the Puro R gene and thereby leave the cells sensitive to puromycin even though the CRISPR/Cas9 had acted on these cells. Moreover, these cells would not have been detected in the sequencing data because PCR amplification employed in those sequencing experiments used a reverse primer that would bind to a portion of the Puro R gene. Therefore, DNA from such cells would not have amplified, and would not have been sequenced. According to this hypothesis, any cells that had the region between the two GFP target sites deleted, would not be selected for by puromycin treatment, which would explain the apparently lower level of enrichment observed relative to that of the Pasha target-containing cells. Moreover, the Myseq reads would have appeared to have a much lower number of reads to the desired mutation.

An alternative hypothesis is that the Pasha smartcode may have a higher level of background and/or cells with this Pasha smartcode in (without any mutations) may have a somewhat basal level of puromycin resistant, i.e. a resistance level that is higher than those cells with the GFP smartcode.

It is apparent that (see Table 4) the FC treatment reduces the background. We have observed that the Pasha smartcode region has many different mutant forms (eg. −1 del. −5 del, −13 del, +2, +5 etc) approx. 15 in a non-pasha targeted population (ie. A GFP targeted population OR a non-Cas9 treated population). This is compared to the GFP smartcode that has only ˜5 mutant forms when not being targeted. When targeted correctly pasha increases to ˜30 mutant forms, this increase is a result of the expected in-frame mutations that are being selected for. The number of GFP mutant forms range from 1-9 depending on selection efficiency (better selection, then low number of mutant forms, as the selection is increasing for “correct” mutant forms).

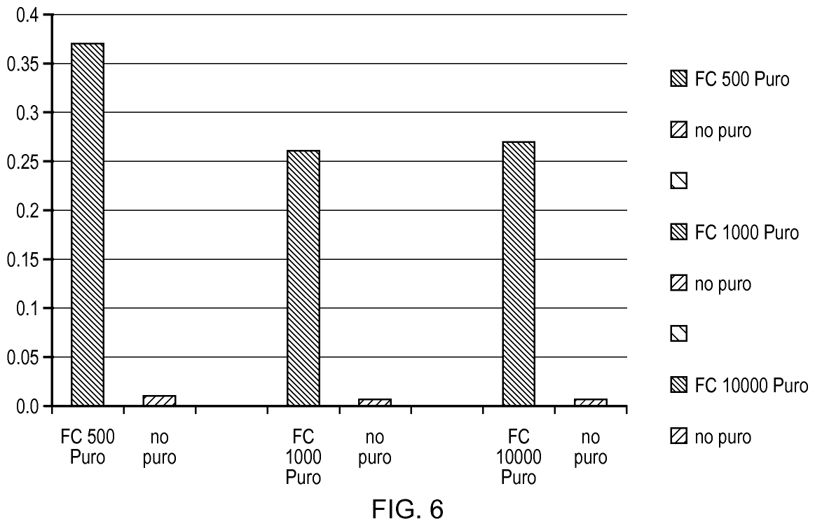

FIG. 6 demonstrates that it is possible to find interesting gene targets/changes in expression of genes within a target cell type simply by comparing a pool that has been selected with puromycin with one that has not had any selection. In particular, a gene of interest will potentially change by 100-fold after puromycin selection. Moreover, FIG. 6 demonstrates that the GFP target cells did exhibit significant enrichment, which would have been expected to be even greater had the deletion of the region between the two GFP target sites not occurred.

The corresponding numerical values for the bars in FIG. 6 are:

| | |
|---|---|
| FC | 500 |
| Puro | 0.37 |
| no puro | 0.0087 |

| | |
|---|---|
| FC | 1000 |
| Puro | 0.26 |
| no puro | 0.0043 |

| | |
|---|---|
| FC | 10000 |
| Puro | 0.27 |
| no puro | 0.002 |

### Example 5—Alternative Smartcode Strategy

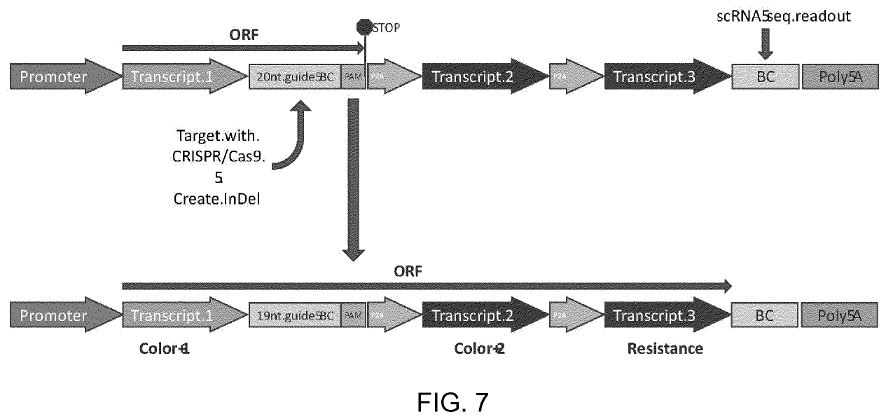

An alternative strategy to that exemplified above is where the smartcode is placed downstream of one or more constitutive transgenes. This strategy protects against leaky scanning and the production of 5′ truncated transgene associated proteins in non-edited cells, which avoidance is something that may be desired in certain circumstances. This is particularly true when the transgene downstream of the smartcode harbors one or more alternative start codons in the 5′ region. In this alternative version, a stop codon is placed downstream of the smartcode, which is in-frame in unedited cells. Located downstream of the stop codon are transgenes for clone selection that in the unedited cells are in, e.g., the −1 reading frame. When the smartcode is edited, the stop codon is driven out of frame, and in those cell where the indel length is 1, 4, 7, etc. the transgenes downstream of the stop codon are driven in-frame, allowing their proper translation (FIG. 7).

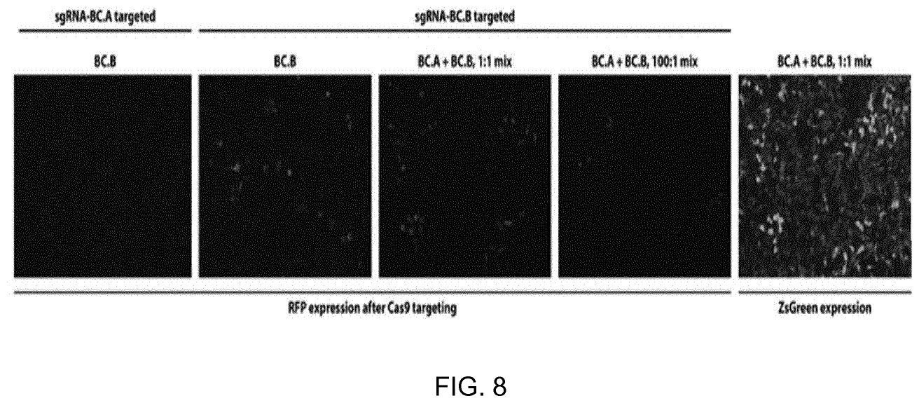

As a proof-of-principal we constructed two vectors following this design, using two independent smartcodes (hereafter called BC.A and BC.B). In this version, the constitutive transcript is ZsGreen and a bicistronic mCherry-P2A-Hygromycin transgene lay downstream of the stop codon. 293T cells were infected separately with the BC.A and BC.B vectors. mCherry positive BC.B infected cells were not visible in non-targeted cells or in cells that were targeted with Cas9 and an sgRNA that targeted BC.A. In contrast, when BC.B infected cells were transfected with Cas9 and an sgRNA targeting BC.B, approximately 25% of the cells became mCherry positive (FIG. 8).

When 1:1 and 1:100 mixtures of BC.B and BC.A cells were transfected with Cas9 and an sgRNA targeting BC.B mCherry positive cells became apparent, and their abundance correlated positively with the number of BC.B cells present in the cellular mixture (FIG. 8).

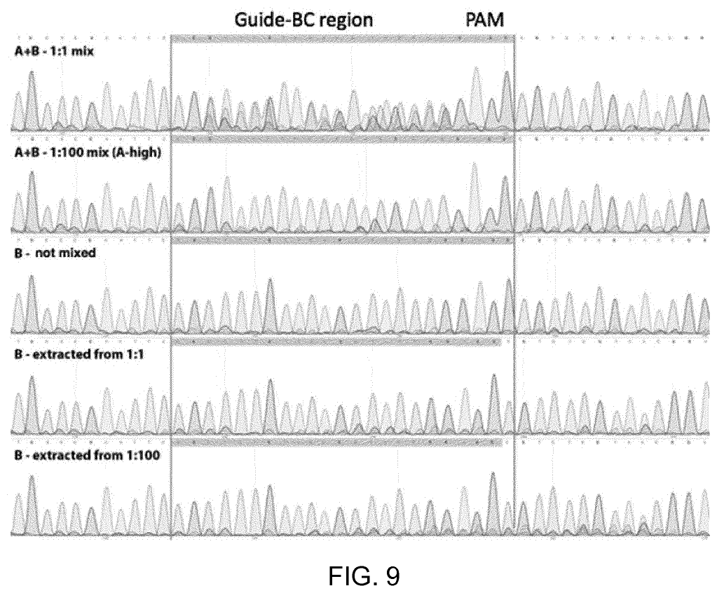

FACS isolation of the mCherry positive cells and subsequent Sanger sequencing of the PCR amplified smartcode construct revealed that these populations represented BC.B infected populations (FIG. 9). In these cells the smartcode was edited to remove a single base at the predicted double strand break site, inside the smart code target (FIG. 9).

For this alternative smartcode strategy, we have also included a second barcode (herein referred to as a SCseq-barcode), which is linked to the smartcode, but lays downstream of the Cas9 activatable transgenes and upstream of a polyadenylation site (e.g. bovine growth hormone polyadenylation signal (BGH)). This placement of the smartcode-linked SCseq-barcode allows for the extraction of single cell transcription profiles for each barcoded cell in a mixed population, using the 10× Genomics single cell sequencing platform. Following single cell library preparation, the cDNA library can be PCR amplified for the transcripts containing the 10× Genomics cellular barcode and the SCseq-barcode. This allows the smartcodes corresponding to each single cell transcriptional profile to be ascertained.

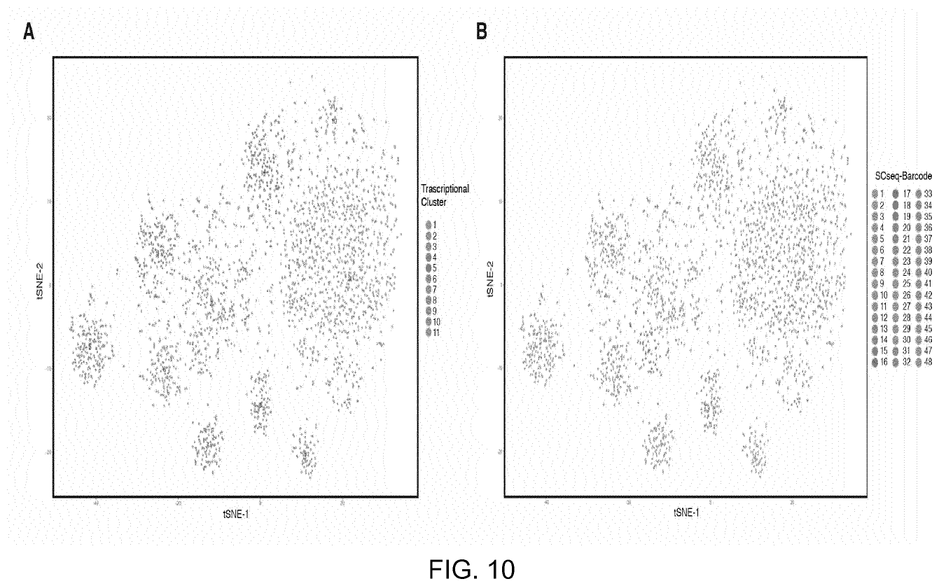

As a proof-of-principal experiment, we inserted a complex barcode population into the SCseq-barcode location into a vector construct that was similar to the one described above, but where the smartcode and stop codon were lacking, and infected the resultant library into 4T1 murine mammary tumor cells. We then FACS isolated 100 single cells and grew them as a pool for a two-week period. At that point 10,000 cells were placed into a 10× Genomics Chromium Single Cell Sequencing machine, which was used to produce a single cell RNA sequencing library. PCR amplification was then applied to extract from the resultant cDNA library both the SCseq-Barcode and the 10× cellular barcode for each of the 10,000 single cells. Following sequencing of both the single cell RNA sequencing library and the aforementioned amplicon, we applied T-Distributed Stoichastic Neighbor Embeded (tSNE) clustering to the transcriptional data, for all cells where there was a corresponding SCseq-barcode that had a greater than 10 times higher representation than any other SCseq-barcode associated with that cell. This analysis produced 11 tSNE clusters (FIG. 10A). We then overlay the SCseq barcode identities of each cell on this plot to demonstrate that individual tSNE clusters represent distinct barcoded populations (FIG. 10B).

All references cited herein are incorporated herein by reference in their entirety and for all purposes to the same extent as if each individual publication or patent or patent application was specifically and individually indicated to be incorporated by reference in its entirety.

The specific embodiments described herein are offered by way of example, not by way of limitation. Any sub-titles herein are included for convenience only, and are not to be construed as limiting the disclosure in any way.

1-83. (canceled)

84. A method of gene editing in a population of cells, comprising incorporating into a single construct at least one guide sequence targeting a genomic loci of interest, and at least one guide sequence targeting a second loci located in the construct or the host genome.

85. The method of claim 84, wherein the second loci comprises a selection marker such that CRISPR-Cas system-mediated editing of said selection marker causes a change in one or more detectable properties of the gene-edited cells, and wherein enrichment of the gene edited cells is based on the change in said one or more detectable properties.

86. The method according to claim 85, wherein editing of the selection marker gives rise to a qualitatively or quantitatively detectable property that distinguishes cells expressing said selection marker from those not expressing the selection marker or expressing said selection marker at a lower level.

87. The method according to claim 86, wherein the qualitatively or quantitatively detectable property is selected from protein fluorescence, products of enzyme reactions, antibiotic resistance, changes in gene sequence, cell surface expression of a protein, or cytotoxicity.

88. The method according to claim 84, wherein providing the populations of gene-edited cells comprises transfecting, infecting or transforming a population of cells with a vector comprising a guide sequence targeting a genomic region of interest and a guide targeting a locus on the same vector.

89. The method according to claim 84, wherein upon selection for editing of the selectable marker, the co-selected genomic locus is edited with high efficiency.

90. The method according to claim 84, wherein the guide sequence targeting the genomic loci is the same or different to the guide sequence targeting the selection marker.

91. The method according to claim 84, wherein upon Cas-mediated editing of the selection marker said selection marker is placed in frame and becomes functional.

92. The method according to claim 84, wherein upon Cas-mediated editing of the selection marker said selection marker is placed out of frame and becomes non-functional.

93. The method according to claim 84, wherein the selection marker comprises a drug selection marker or fluorescent protein or cell surface marker.

94. The method according to claim 84, wherein the at least one selection marker is a drug selection marker that upon Cas-mediated editing is placed in frame and becomes functional.

95. A vector encoding:
(i) a single guide RNA (sgRNA) targeting an endogenous locus; and
(ii) an sgRNA targeting a second locus.

96. The vector according to claim 95 wherein (ii) is an sgRNA able to activate, by inducing a frame shift, a selectable marker.

97. The vector of claim 95, wherein the selectable marker is a drug selection marker that, upon editing, is placed in frame and becomes functional.
